(19) United States
(12) Patent Application Publication
(10) Pub. No.: US 2003/0153936 A1
(43) Pub. Date: Aug. 14, 2003

(54) Tourniquet instrument

(76) Inventor: Rizk El-Galley, Birmingham, AL (US)

Correspondence Address: BRADLEY ARANT ROSE & WHITE, LLP, INTELLECTUAL PROPERTY DEPARTMENT-NWJ, 1819 FIFTH AVENUE NORTH, BIRMINGHAM, AL, 35203-2104, US

(21) Appl. No.: 10/073,494

(22) Filed: Feb. 11, 2002

(51) Int. Cl. A61B 17/08

(52) U.S. Cl. 606/158

(57) ABSTRACT

A surgical instrument that allows a surgeon to temporarily halt the flow of blood to a tissue during a surgical procedure (temporary hemostasis) and restore the flow of blood to the tissue when desired without damaging the tissue is described. The instrument comprises a handle means, a hollow barrel member, a loop and an operating means coupled to the loop to drive the loop between contracted state and an expanded state. Preferably, one end of the loop is removable from the operating means so that temporary hemostasis can be induced in virtually any tissue of the body.

### FIELD OF THE DISCLOSURE

The present disclosure relates generally to an improved device for hemostasis in tissues of the body via the use of a tourniquet instrument during surgery, especially during laparoscopic and endoscopic surgical procedures.

### BACKGROUND

Laparoscopy is a technique for performing a surgical operation on a patient without requiring that a major incision be made. As a result, laparoscopic procedures are considered minimally invasive. The advantages of these minimally invasive procedures include decreased recovery times, decreased hospital stays, decreased pain, decreased infection rates and decreased cost both for the consumer and the health system as a whole. A number of surgical procedures can now be performed via laparoscopy, such as cholecystectomies, acid reflux procedures, removal of small tumors, cardiomyotomy, restriction of the small bowel, lysis of adhesions, electrocoagulation, colectomy, appendectomy, hernia repair, and splenic surgery, just to name a few. As the tools and technology available for laparoscopic procedures becomes more advanced, the list of surgical procedures is growing.

In general, laparoscopic operative technique involves several phases. First, a pneumoperitoneum is established. Second, trocars are inserted into the in the body (through a minor incision) for the introduction of the camera and surgical instruments. A spring-loaded needle with a blunt tip is inserted through one of these small incisions to initiate the pneumoperitoneum. The patient is placed in Trendelenburg's position and insufflation of CO₂ is begun until an intraperitoneal pressure in the range of 12 to 18 mmHg is attained. The needle is then removed and replaced with a trocar through which a videoscope is inserted. After inspecting the peritoneal cavity for trauma caused by the initial procedures, the remaining trocars are inserted under direct laparoscopic observation. The different surgical instruments needed to complete the procedure may then be inserted through the trocars. After the procedure is completed, the trocars are removed and the incisions closed.

There are several laparoscopic devices for ligating and/or constricting tissue. Such devices are commonly used when it is desired to permanently stop the flow of blood to a tissue, and in many cases are used in the place of sutures in laparoscopic surgery. In addition, these devices can be used when it is desire to sever a tissue as in the tubal ligation procedure. In general, these devices utilize a hollow plastic tube containing a pre-formed loop of suture material with a slip knot at the terminal end. The alternate end of the suture material is connected to a plastic handle that extends out of the hollow tube. The surgeon pulls the handle which closes the loop around the tissue to be ligated. The hollow tube has a tapered distal end that forces the slip knot to tighten around the tissue, causing strangulation of the tissue within the loop. After the surgeon is satisfied the knot is in place, the excess suture material adjacent to the slip knot is removed. Such devices have been used to ligate/constrict severed blood vessels, appendix stumps and similar tissues in the body.

However, use of these devices has several limitations. First, the tissue to be ligated must have a free end so that the preformed loop can be placed over the tissue (for example, in ligating a blood vessel that has been severed during the surgical procedure), or the tissue must be of a structure so that a preformed loop can be placed over a portion of the tissue (for example, in the tubal ligation procedure). Secondly, the devices are not meant to be used when only temporary hemostasis is desired, such as the temporary cessation of the flow of blood to a tissue. Therefore, there is a need for a surgical device that can be used for temporary hemostasis of tissues and that can be used on any tissue, regardless of the structure of the tissue.

The present disclosure is directed to a surgical instrument allowing a surgeon to quickly and efficiently provide temporary hemostasis to tissues during surgical procedures, such as laparoscopy. This modification allows the surgeon to provide temporary hemostasis to tissues that previously would not be subject to such a procedure, especially during laparoscopic surgical procedures.

### SUMMARY

The surgical instrument of the present disclosure an instrument that allows a surgeon to temporarily halt the flow of blood to a tissue (referred to as temporary hemostasis) and restore the flow of blood to the tissue when desired without damaging the tissue. Although the modified instrument of the present disclosure is particularly well suited to laparoscopic and endoscopic procedures, it may also be used in conventional surgical procedures. Previous laparoscopic devices are not designed to induce temporary hemostasis, but are designed to ligate tissue in the same manner as a suture. Importantly, using the instrument of the present disclosure the tissue is not damaged during the induction of temporary hemostasis.

The instrument comprises a handle means, a hollow barrel member with a proximal end, a distal end, an opening at the distal end, a loop and an operating means to drive the loop between contracted state and an expanded state. The loop is functionally coupled to the operating means. When the loop is in the contracted state, hemostasis of a tissue is induced. Hemostasis is relieved and normal blood flow restored when the loop is driven to the expanded state. The loop is designed so that it is removable from the operating means. Therefore, a loop can be formed around almost any tissue in the body. Previous devices that were designed to ligate tissues could only ligate tissue with a free end, or tissue that was of an architecture so that a preformed loop can be placed over a portion of the tissue. As a result of using the instrument of the present disclosure, trauma to the patient is reduced and the time to complete the surgery is reduced. As a result, cost to the patient and to the health system in general are decreased. Additional advantages of the invention will become apparent to those of ordinary skill in the art from the description that follows, when read in conjunction with the attached figures.

### BRIEF DESCRIPTION OF THE DRAWINGS

FIG. 1A is a side view illustrating one embodiment of the surgical device of the present disclosure;

FIG. 1B is a side view illustrating an alternate embodiment of the surgical device of the present disclosure

FIG. 2A is a side view of one embodiment of the operating means and the loop means of the surgical device of the present disclosure;

FIG. 2B is a top view of one embodiment of the operating means and the loop means of the surgical device of the present disclosure;

FIG. 2C is a cross section of the loop means taken along line 2C of FIG. 2B;

FIG. 3 is a side view of one embodiment of the barrel member incorporated into the surgical device illustrated in FIGS. 1A and 1B;

FIG. 4A is a detailed top view of the distal end of the barrel member illustrated in FIG. 3; and

FIG. 4B is a detailed front view of the distal end of the barrel member illustrated in FIG. 3.

### DETAILED DESCRIPTION

It is to be understood that although this disclosure will refer to surgical procedures or surgery, these terms will be understood to include laparoscopic procedures, endoscopic procedures, as well as other surgical procedures where tissue hemostasis may be employed. The term hemostasis means the arrest of bleeding or blood flow either by constriction or other surgical means. The term tissue, or similar terms, means any organ or structure of the body that can be subject to hemostasis, including but not limited to, the liver, the spleen, the large and small intestines, blood vessels and appendix stumps. The term “contracted state” when referring to the position of the loop means that the loop is contracted around a tissue in a manner sufficient to effectively induce hemostasis to the tissue. The term “expanded state” when referring to the position of the loop means that the loop is released from a tissue a sufficient amount so that hemostasis is relieved and blood flow is restored to the tissue.

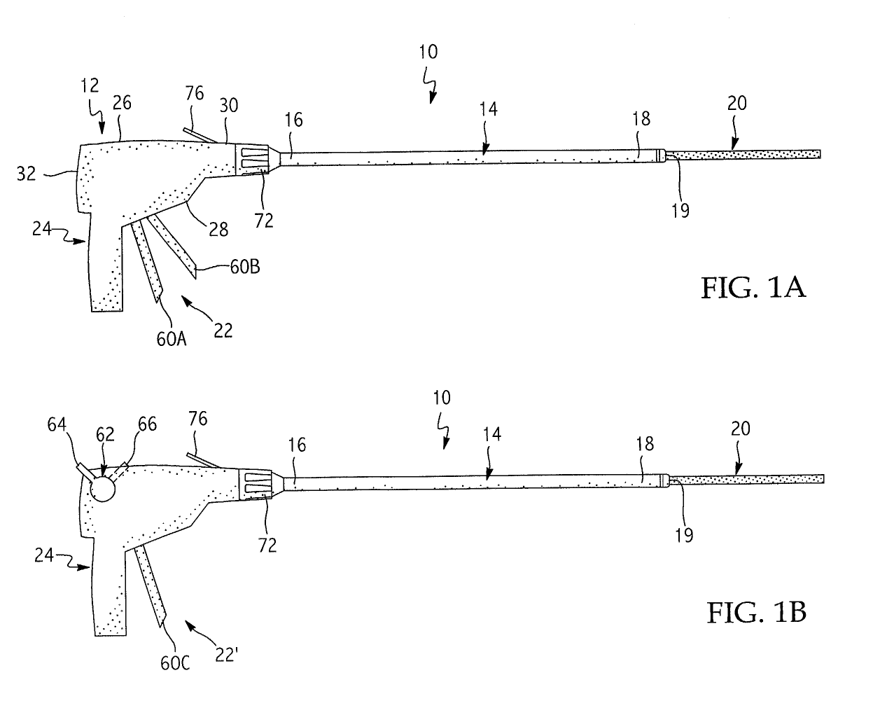

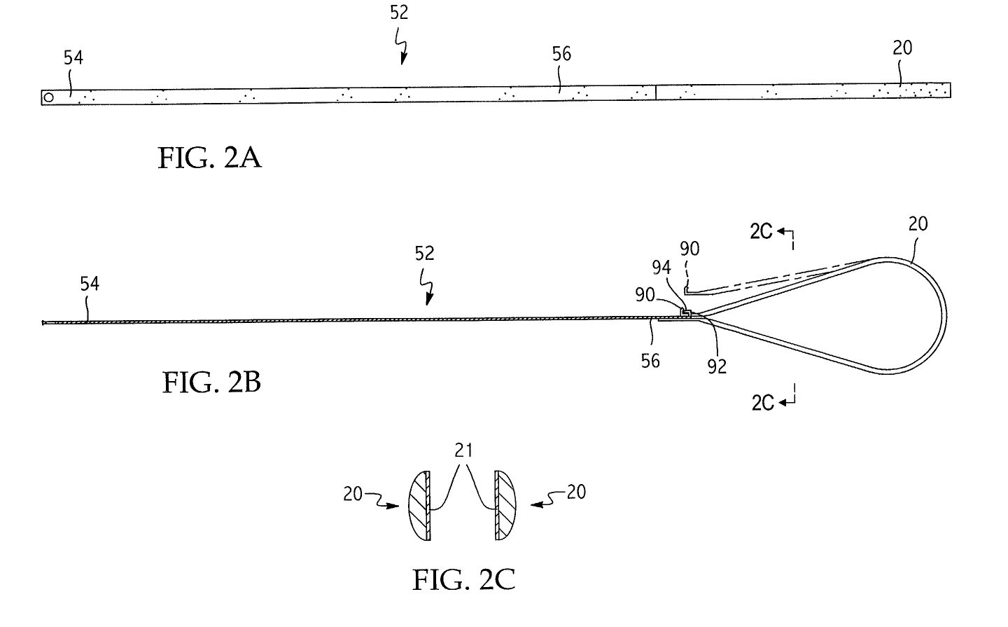

The device of the present disclosure is illustrated in FIGS. 1-4. With reference to FIG. 1A, the instrument 10 comprises a handle means 12, a hollow barrel member 14, the barrel member 14 having a proximal end 16, a distal end 18, and a opening 19 at the distal end 18, a loop 20 and an operating means (illustrated in greater detail in FIGS. 2A and 2B). The loop 20 is capable of alternating between a contracted state and an expanded state in response to manipulation of the operating means, and is functionally coupled to the operating means. The handle portion 12 provides a means for the surgeon to grasp the instrument in a convenient manner so that the surgeon can manipulate the instrument and to house the operating means and other component of the instrument 10. The design of the handle portion can vary to accomplish these objectives and such variation is well within the ordinary skill in the art of one in the field. In the embodiment described, the handles means 12 further comprises a grip 24, an upper side 26, a lower side 28, a first end 30 and a second end 32.

The operating means 22 serves alternate the loop between the contracted state (where temporary hemostasis in a tissue is induced) and the expanded states (where blood flow is restored to the tissue). The operating means can take on several forms. In the embodiments illustrated, the operating means comprises a ratchet means (not shown) of standard design, a drive band 52, the drive band 52 having a first end 54 and a second end 56, and an activating means 22, with the ratchet means (not shown) being operationally linked to the activating means 22 and the first end 54 of the drive band 52. The second end 56 of the drive band 52 is linked to the loop 20. Preferably, the activating means 22 is at least one trigger 60, although other means such as buttons or wheels could be used. In the embodiment of the activating means 22 illustrated in FIG. 1A, the activating means 22 is shown as a triggers 60A and 60B. In this embodiment, trigger 60A is a contract trigger and trigger 60B is a release trigger, such that manipulating the contract trigger 60A causes the ratchet means (not shown) to slide the drive band 52 away from the distal end 18 of the barrel member 14, causing the loop 20 to contract until the loop 20 is in the contracted state, and manipulating the release trigger 60B causes the ratchet drive (not shown) to slide the drive band 52 toward the distal end 18 of the barrel member 14 causing the loop 20 to expand until the loop 20 is in the expanded state.

In an alternate embodiment illustrated in FIG. 1B, the activating means 22′ may employ only one trigger 60C. In this case the operating means further comprises a direction control 62 to regulate the action of the ratchet means (not shown), with the direction control 62 functionally linked to the ratchet means (not shown) and the trigger 60C. It is preferred that the direction control 62 be capable of alternating between at least two states so that the activating means 22′ can cause the loop 20 to alternate between the contracted state and the expanded state. In operation, manipulating trigger 60C causes the ratchet means (not shown) to slide the drive band 52 away from the distal end 18 of the barrel member 14 causing the loop 20 to contract until the loop 20 is in the contracted state when the direction control 62 is set to a contract state (illustrated as 64 in FIG. 1B), and manipulating trigger 60C causes the ratchet means (not shown) to slide the drive band 52 toward the distal end 18 of the barrel member 14 causing the loop 20 to expand until the loop 20 is in the expanded state when the direction control 62 is in the release state (illustrated in phantom as 66 in FIG. 1B). The positions 62 and 64 may also be reversed, such that when the direction control 62 is in position 64, manipulation of the activating means 22′ causes the loop 20 to be in the expanded state and vice versa.

The barrel member 14 is a hollow elongate structure with an internal central passage 70 extending substantially the length of the barrel member 14. The shape of the barrel member is of a generally circular cross section and of a dimension sufficient to house the components of the operating means and a first channel 74 (described below). The length of the barrel member 14 will vary depending upon the use of the instrument 10. If the instrument 10 is used for laparoscopic procedures, the barrel member 14 will be of sufficient length for such procedures. The barrel member 14 is operationally coupled to the handle means by any convenient method. In the embodiments illustrated, the proximal end 16 of the barrel member 14 is removably coupled to the first end 30 of the handle means 12 by a lock connector 72. Connected in this manner, barrel member 14 is removable from handle means 12. The barrel member 14 houses at least some of the components of the operating means. The proximal end 18 of the barrel member 14 may optionally comprise a guard 78 that at least partially spans the diameter of opening 19. As illustrated in FIGS. 1 and 4A, the guard 78 divides the opening 19 of barrel member 14 into a left entry channel 80 and a right entry channel 82. The function of the guard 78 is to prevent tissue being subject to hemostasis from being damages by being pressed into opening 19 when the tissue is being contracted by loop 20.

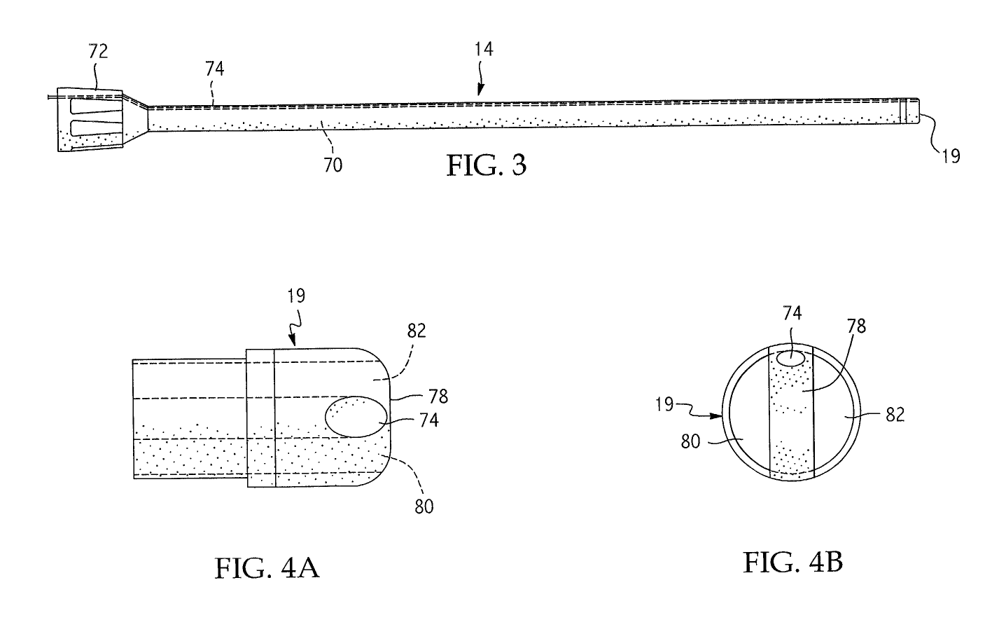

The central passage 70 may further comprise a first channel 74 inside internal central passage 70. The first channel 74 is connected to an entry port 76 and an exit port 78. In the embodiment illustrated in FIGS. 3A and 3B, the entry port is located on the upper portion 26 of handle means 12 and the exit port is located on the distal end 18 of the barrel member 14. The first channel 74 can be used for a variety of purposes, including, but not limited to, the delivery of medicaments to tissue subject to hemostasis. The medicaments to be delivered can be of any variety, including, but not limited to, coagulants, antibiotics and growth stimulators, however, a preferred medicament is fibrin. The form of the medicament is preferably a liquid, however, medicaments in a gel form may also be applied. The medicaments are preferably delivered by applying air pressure to the entry port 76 such that the medicaments are forced from the exit port 78. The first channel 74 is if a sufficient diameter to deliver the medicaments, but a preferred diameter is in the range of about 1 mm to 1.5 mm in diameter.

The loop 20 is attached to the second end 56 of the drive band 52. The loop 20 extends out of the opening 19 of barrel member 14. As the loop 20 is contracted or expanded through manipulation of the operating means, less or more, respectively, of the loop 20 extends through the opening 19. At least one end of the loop 20 is preferably reversible connected to the second end 56 of drive band 52 by an attachment means. By being reversibly secured, at least one end of the loop 20 may be free so that the loop 20 may be placed around virtually any tissue without the need for the tissue to have a free end. The exact mechanism of attachment is not critical. In the embodiment shown in FIG. 2B, the attachment means is illustrated using a pressure connection. One end of loop 20 comprises a lip 90. The loop 20 is inserted into a receiving means, illustrated as bracket 92. As the loop 20 is inserted, the lip 90 engages a shoulder 94 on bracket 92 such that loop 20 is secured in bracket 92. When it is desired to remove loop 20 from bracket 92, a pulling force can be applied to loop 20 such that the lip 90 passes under the shoulder 94 on bracket 92. Other means to removably secure the loop 20 to the second end 56 of drive band 52 may be used. Alternatives include, but are not limited to, placing a metal tip on one end of the loop 20 that is removably received by a receiving means on the drive band 52, such as a bracket or opening. The metal tip on the loop 20 and/or all or a portion of the drive band 52 may be magnetic to aid in the initial attachment. Alternately, the loop 20 may be permanently secured to the second end 56 of the drive band 52.

The loop 20 can be manufactured from various materials, so long as the function of providing temporary hemostasis to tissues without damaging the tissue is maintained. Examples of preferred materials are wire, rubber, elastomeric materials or composite materials. In addition, the inner surface of the loop 20 may comprise a textured surface 21 so that the loop 20 may easily grasp the tissue of interest. In addition, the width and thickness of the loop 20 may be varied as desired depending upon use, and such determinations are well within the ordinary skill of one in the art.

The following provides an example of how one embodiment of the instrument 10 might be used during a laparoscopic procedure. This example should not be interpreted as limiting the use of the instrument 10 solely to laparoscopic procedures, but as an illustration of its operation. The instrument 10 is placed through a trocar tube so that at least a portion of the barrel member 14 is within a body cavity, such as the abdomen, of a patient. When it is desired to apply temporary hemostasis to a tissue during the procedure, the surgeon manipulates instrument 10 so that loop 20 is placed over the tissue of interest. If the tissue does not have a free end, or the tissue is not of a structure so that the loop 20 can be placed over a portion of the tissue, one end of the loop 20 may be removed from the drive band 52 with a laparoscopic grasper or similar instrument and placed around the tissue of interest. Once in place the free end of the loop 20 may be secured back on the drive band 52. Once the loop 20 is in place, the surgeon manipulates the activating means 22 to drive the loop 20 to the contracted state, thereby inducing temporary hemostasis in the tissue. The desired procedure is performed, and once completed, the surgeon manipulates the activating means 22 to drive the loop to the expanded state and restore blood flow to the tissue. If desired, medicaments (such as fibrin) can be dispensed through the first channel 74 to the tissue before, during or after temporary hemostasis is induced.

The method described above has many advantages over current methods for forming sutures in laparoscopic procedures. First, since at least one end the loop 20 is removable, hemostasis can be induced in virtually any tissue in the body. The at least one end of the loop can be removed from the end of the drive band and replaced through the use of available instruments. Second, the instrument of the present disclosure provides a convenient means or inducing temporary hemostasis in a tissue without damaging the tissue. In addition, the instrument allows the surgeon to apply medicaments to tissues subject to hemostasis. Fourth, the instrument of the present disclosure is simple to use requiring minimal retraining for the surgeon to become proficient in its use. Finally, the process of inducing hemostasis can be performed quickly and accurately, allowing the surgeon to respond to unanticipated events that would before require the laparoscopic procedure be terminated and conventional surgery initiated.

I claim:

1. A surgical instrument for use in surgical ligation, the instrument comprising:
a. a handle means;
b. a loop capable of alternating between a contracted state and an expanded state;
c. an operating means functionally and removably coupled to the loop such that the operating means can cause the loop to alternate between the contracted state and the expanded state; and
d. a hollow barrel member comprising a central passage, the barrel member operationally coupled to the handle means and housing at least a portion of the operating means and the loop.

2. The instrument of claim 1 where the operating means comprises an activating means, a ratchet means and a drive band, the ratchet means being functionally linked to the activating means and the first end of the drive band, with the loop means functionally and removably coupled to the second end of the drive band.

3. The instrument of claim 2 where the activating means comprises at least one trigger.

4. The instrument of claim 3 where the activating means comprises a contract trigger and a release trigger, such that engaging the contract trigger causes loop means to be in the contracted state and engaging the release trigger causes the loop means to be in the expanded state.

5. The instrument of claim 2 where the operating means further comprises a direction control operationally linked to the activating means, the direction control capable of alternating between at least two states.

6. The instrument of claim 5 where the activating means comprises one trigger, and manipulating the trigger causes the loop means to be in the contracted state when the direction control is set to a contract state, and depressing the release trigger causes the loop means to be in the expanded state when the direction control is set to a release state.

7. The instrument of claim 1 where the instrument further comprises an entry port, an exit port and a first channel within the central passage, the first channel connecting the entry port and the exit port.

8. The instrument of claim 7 where the entry port, the exit port and the first channel are used to deliver medicaments to tissues in the body subject to the surgical ligation.

9. The instrument of claim 8 where the medicament is fibrin.

10. The instrument of claim 1 where the loop is an elastic band, the elastic band comprising a textured interior surface.

11. The instrument of claim 4 where the loop is an elastic band, the elastic band having a textured interior surface.

12. The instrument of claim 6 where the loop is an elastic band, the elastic band having a textured interior surface.

13. The instrument of claim 1 where the handle means comprises a grip, an upper side, a lower side, a first end and a second end.

14. The instrument of claim 13 where the barrel member is operationally coupled to the first end of the handle means by a lock connector, the lock connector and the first end of the handle means having complementary engaging means to facilitate the coupling.

15. The instrument of claim 13 where the entry port is located on the upper side of the handle and the exit port is located on the distal end of the barrel member.

16. The instrument of claim 1 where the loop means is removably secured to operating means by an attachment means.

17. The instrument of claim 16 where the attachment means comprises a pressure connection

18. The instrument of claim 2 where the pressure connection further comprises an interaction between a lip on at least one end of the loop and a receiving means on the drive band.

19. The instrument of claim 2 where the attachment means comprises an interaction between a metal tip on at least one end of the loop and a receiving means on the drive band.

20. The instrument of claim 19 where the metal tip and or the receiving means is magnetic.
